(19) United States
(12) Patent Application Publication
(10) Pub. No.: US 2002/0164362 A1
(43) Pub. Date: Nov. 7, 2002

(54) Cosmetic product containing shellac

(76) Inventors: Karin Golz-Berner, Monaco (DE); Leonhard Zastrow, Monaco (DE)

Correspondence Address: COLLARD & ROE, P.C., 1077 Northern Boulevard, Roslyn, NY, 11576-1696, US

(21) Appl. No.: 10/081,025

(22) Filed: Feb. 21, 2002

Related U.S. Application Data
(63) Continuation-in-part of application No. 09/463,877, filed on Jan. 31, 2000, now abandoned.
Application No. 09/463,877 is a 371 of international application No. PCT/DE98/02086, filed on Jul. 21, 1998.

(30) Foreign Application Priority Data
Aug. 1, 1997 (DE) DE 197 34544.1

(51) Int. Cl. A61K 7/00

(52) U.S. Cl. 424/401

(57) ABSTRACT

The invention relates to cosmetic emulsion or a gel that contains shellac. In accordance with the invention, a shellac-containing cosmetic product is produced which contains shellac in the aqueous phase. The proportion of pure shellac lies between 0.1 and 20 wt. % in an O/W emulsion; between 0.1 and 15 wt % in a W/O emulsion; and between 0.1 an 10 wt % in a hydrogel. Between 1 and 20 wt. % of other cosmetically active components or other supporting materials are contained in the oil phase or the aqueous phase, or in both phases. The invention also relates to a method for producing the cosmetic product. In this method, in order to produce the aqueous shellac solution or dispersion that is to be used, 0.1-3 wt. % of a water-soluble film former and 0.1-1 wt. % of an acid-resistant gel former with a pH of 2-4.2 are added.

### The invention concerns a cosmetic emulsion or a gel containing shellac.

It is known that shellac has been used as a resin component in nail polish as well as cosmetic hair sprays, hair setting lotions, and shampoos. Shellac has been used as a film-forming substance in alcoholic compositions. Shellac has been mostly replaced by a number of modern copolymers that fulfilled this function better.

Nevertheless, shellac is still used for certain applications such as hair spray or shampoo, not least of all because it is a very pure natural product that is available in good quality.

In addition, it is known from International Patent WO96/41630 that shellac can be used as a separating agent between layers that have separated from one another in a sunscreen preparation consisting of three or more layers, so that a multilayer structure of the sunscreen on the skin is formed, and the skin comes in contact only with the layer containing inorganic sunscreen agents that are tolerated well by the skin.

The object of this invention is to improve the feeling of traditional emulsions and gels on the skin by avoiding an oily or sticky feeling while at the same time improving its waterfastness and achieving stable emulsions and gels. It has been found that addition of an aqueous shellac dispersion to O/W or W/O emulsions or hydrogels may under certain conditions lead to a cosmetic product (emulsion or gel) having a different type of structure and thus also different properties.

The prerequisite for preparation of such emulsions and gels is the use of pure aqueous shellac solutions or dispersions. Shellac is normally soluble only in solvents such as alcohols, ketones and organic acids. Shellac is insoluble in hydrocarbons, and is soluble in water only in the form of saponified shellac. It has been found that unsaponified shellac is also up to approximately 30 wt % soluble in water under certain conditions, and dispersions in water containing up to approximately 60 wt % are possible. Such solutions/dispersions consist of

shellac in the range of 1 to 60 wt %, preferably 10 to 60 wt %, especially 15-60 wt %;

a water-soluble film-forming agent in the range of 0.1 to 3 wt %;

a gel-forming agent that is stable in the acidic range, in the range of 0.1 to 1 wt %;

an acid to adjust the pH to a value of 2 to 4.2, preferably 2 to 3.5;

and water up to 100 wt %.

These solutions or dispersions are prepared by placing solid, purified, dewaxed shellac in an aqueous acidic solution of the film-forming substance and the gel-forming substance at a pH of 2 to 4.2, while stirring at a temperature in the range of 5° C. to 20° C., and then homogenizing the ingredients of the solution or dispersion together well.

Thus, this invention makes available a cosmetic product containing shellac, comprising a pure shellac content in the aqueous phase, wherein the shellac component is

in the range of 0.1 to 20 wt % in an O/W emulsion,

in the range of 0.1 to 15 wt % in a W/O emulsion,

in the range of 0.1 to 10 wt % in a hydrogel,

and the oily phase or the aqueous phase or both phases may contain additional cosmetic active ingredients or vehicles in the range of 1 to 20 wt %. All amounts here are based on the weight of the total composition.

The new cosmetic product permits an especially smooth distribution on the skin without having an oily effect, produces a very pleasant soft feeling on the skin and is not sticky.

Another object of this invention is the above cosmetic product containing shellac, comprising a pure shellac content in the aqueous phase, wherein the shellac component

is in the range of 0.1 to 20 wt % in an O/W emulsion,

is in the range of 0.1 to 15 wt % in a W/O emulsion,

is in the range of 0.1 to 10 wt % in a hydrogel,

and the oily phase or the aqueous phase or both phases may contain additional cosmetic active ingredients or vehicles in the range of 1 to 20 wt %,

prepared by

a) preparing an emulsion or a hydrogel in the usual way by mixing a primary emulsion phase, containing the cosmetic active ingredients and additives provided therein, with a secondary emulsion phase, containing the cosmetic active ingredients and additives provided therein;

or mixing a gel-forming substance with water and adding the cosmetic active ingredients and additives provided therein; and

b) preparing a 5 wt % to 60 wt % aqueous shellac solution or dispersion by mixing pure shellac dry solids having a molecular weight of approximately 1000 to 1010 with water in the presence of a water-soluble film-forming substance and a gel-forming substance that is stable in an acidic pH range at a temperature of 5° C. to 20° C. and a pH of 2 to 4.2; and

c) heating the emulsion or the gel from step a) to a temperature in the range of 35° C. to 45° C. while stirring; and

d) adding the shellac solution or dispersion from step b) to the former while stirring at 150 to 1000 rpm for a period of 5 to 60 minutes at 20° C. to 45° C.; and then

e) cooling the resulting product to ambient temperature.

For example, a polyvinylpyrrolidone such as PVPK30 may be used as the water-soluble film-forming substance. A further possible substance is chitosan, microcrystalline chitosan, quaternary chitosan, polymers of the acrylic acid series, quaternary cellulose derivatives, hyaluronic acid and salts of it. Mixtures of the mentioned substances may be used.

A gel-forming substance that is stable in the acid range is a modified natural polysaccharide such as guar hydroxypropyltrimonium chloride (Jaguar C14S®). A further possible gel is agar, alginates, alginic acid. Mixtures of the mentioned gels may be used.

The shellac used here is a processed product. Shellac is a natural resin excreted by the female insect Laccifer lacca Kerr. The purified, wax-free resin, which has a molecular weight of approximately 1000 and is used for cosmetic purposes, consists of a number of hydroxymonodicarboxylic acids in the form of lactones, lactides and intramolecular esters. The main components are aleuritic acid, shellolic acid and jalaric acid (C₁₅H₂₀O₅) with molecular weights of 304, 296 and 280. Shellac is therefore an oligomer of hydroxycarboxylic acids rather than a polymer. Impurities can be removed by a solvent extraction method, e.g., with an alcohol, and after decolorizing with activated carbon and evaporating the alcohol, the shellac can be converted to a yellowish to faintly yellowish solid. Such a solid is the starting material for the aqueous solutions and dispersions used with this invention.

Use of shellac in pure form as an aqueous solution or dispersion with a viscosity of >100 cP·s in aqueous cosmetic products is made possible for the first time through the present invention in an advantageous manner, especially in O/W emulsions.

The shellac solutions/dispersions used have a viscosity of approximately 500 cP·s at a shellac content of approximately 10 wt %, approximately 1000 cP·s at 20 wt %, and approximately 2500 cP·s at 35 wt %. Shellac solutions or dispersions of approximately 400-500 cP·s and higher are preferred. Thus, dispersions of 55-60 wt % shellac have viscosities of approximately 100,000 cP·s, are creamy to pasty and can be processed well with the corresponding equipment. Because of the dependence of viscosity on the concentration of shellac in aqueous dispersions and solutions, it is expedient to work with viscosities in practical implementation of the invention.

With an O/W emulsion, the structure of an emulsion according to this invention is presumably such that the shellac is distributed in the continuous aqueous phase, or with a W/O emulsions, water droplets are surrounded by a layer of the natural resin, or in a hydrogel, shellac droplets are in a random distribution in the gelled aqueous phase.

The shellac content in the emulsion or hydrogel is in the range of 0.5 to 20 wt %, preferably in the range of 0.5 to 8 wt %.

In the O/W emulsion, the oil content is in the range of 3 to 20 wt %, preferably 3.5 to 7.7 wt %, and the water content is in the range of 50 to 80 wt %, preferably 50 to 65 wt %.

In the W/O emulsion, the water content is in the range of 40 to 65 wt %, preferably 40 to 55 wt %, and the oil content is in the range of 5 to 20 wt %, preferably 4.5 to 10 wt %.

In the hydrogel, the water content is in the range of 40 to 80 wt %, preferably 50 to 80 wt %.

The oily phase or the aqueous phase or both phases may contain additional cosmetic active ingredients and cosmetic additives or vehicles.

Cosmetic active ingredients include, for example, emulsifiers, such as 1-decane homopolymer, polyglycerol esters and other known emulsifiers for W/O emulsions, in addition, C12-C15 alkyl benzoate, beeswax, steareth, shea butter and other known O/W emulsifiers.

Other possible additives include isopropyl myristate, isopropyl palmitate, isopropyl stearate, carbomer, cetearyl alcohol, lecithin, copolymers, paraffin oil, cetyl alcohol, propylene glycol, polyglycol, jojoba oil, silicone oil, coconut oil, palm oil, mineral oil, cetyl palmitate, acrylates, C10-C30 alkyl acrylate crosspolymer, magnesium aluminum silicate, hydroxyethyl-cellulose and other substances with which those skilled in the art are familiar and which are adapted to the specific applications such as lipstick, eye makeup, other cosmetics, etc.

As additional active ingredients, the preparation may contain 1,3- and 1,6-β-glucan, CM-glucan®, allantoin, TiO₂, ZnO, ZrO₂, SiO₂ and additional UVA and UVB blocking substances such as organic sunscreens. For example, benzophenone derivatives, 3-benzylidene camphor derivatives, esters of cinnamic acid or salicylic acid, 4-aminobenzoic acid derivatives, such as benzophenone-3, butylmethoxydibenzoylmethane, octyl methoxycinnamate, octyl salicylate and also water-soluble products such as phenylbenzimidazolesulfonic acid or benzophenone sulfonic acid derivatives may be used for this purpose. Combinations with UVA filters are also possible.

An emulsion according to this invention may also contain powdered substances selected from the group consisting of pigments such as metal oxides (iron oxides, TiO₂, ultramarine blue, chromium oxide, mica-titanium oxide, mica-iron oxide, mixtures thereof) and also other powdered substances such as Sio₂, silica, ZnO, kaolin, SiO₂-modified kaolin (according to WO96/17588), polytetrafluoroethylene, nylon, talc, mica, polymethyl methacrylate, polyethylene, natural organic compounds such as encapsulated or unencapsulated grain starch and mixtures thereof.

In addition, cosmetically beneficial additives that can also be used include water, monohydric and polyhydric alcohols, such as ethanol, isopropanol, propylene glycol, glycerol, ethylene glycol, C₉-C₁₁ alcohols, etc.; methyl, ethyl or butyl ether with ethylene glycol, propylene glycol or diethylene glycol; gel-forming substances; vitamins such as vitamin A, E, B₂, B₆, B₁₂, C; panthenol, aloe vera, allantoin, bisabolol; coloring agents; preservatives; protective agents, radical scavengers; enzymes, plant ingredients; natural anti-inflammatory agents; moisturizers; antioxidants; pH regulators; pigments such as soluble melanin; perfume; copper gluconate, etc.

In addition, the emulsion or the gel may contain oxygen-loaded asymmetrical lamellar aggregates according to WO94/00109 or dispersed or magnetically hard, single-crystal particles contained in the asymmetrical lamellar aggregates or in liposomes and having a high coercive field strength according to WO95/03061. Digestion products of yeasts or plant substances produced by a gentle ultrasonic digestion method according to WO94/13783 are also very advantageous.

The invention has the following advantages:

1. Incorporating shellac into emulsions or hydrogels leads to an improved feeling on the skin, which has been proven by tests,

2. Adhesion to the skin is improved for decorative cosmetics, i.e., lipstick, tinted daily creames, makeup-cosmetics, powder and all products containing pigments and pigment-like substances such as magnetically hard single-crystal particles of barium or strontium hexaferrite (see WO95/03061).

3. Stability in water is increased in sun care products, i.e., sun lotions, sun emulsions, after-sun care lotions, sun gels, etc.

4. In sun care products it acts as an anti-salt water/anti-sand agent, because it does not leave any oily film on the skin.

5. No stickiness on the skin is detected with any products.

6. Homogeneous emulsions are also obtained with large amounts of inorganic pigments such as Tio₂ or ZnO or combinations of TiO₂, ZnO and SiO₂, which is advantageous for sun care products with a high sunscreen factor in particular.

7. An especially permanent (long-lasting) product is achieved with lipstick and lip gloss with a special gloss that can be described as matt brilliance.

The invention will be explained in greater detail below on the basis of examples. All amounts are based on weight unless explicitly stated otherwise.

### EXAMPLE 1

Preparation of an O/W Emulsion

An O/W emulsion was prepared from the following ingredients. Phases A and B were prepared separately at 60° C. while stirring and were combined at the aforementioned temperature and then cooled to 40° C. Phases C and D were added to the total mixture while stirring.

| Phase A | |
|---|---|
| C12-C15 alkyl benzoate | 3.5% |
| Steareth | 1.5% |
| Phase B | |
| Water | q.s. ad 100 |
| Glycerol | 1.5% |
| Coloring pigments | 2.0% |
| Phase C | |
| Babassu oil | 2.5% |
| Silicone oil | 1.5% |
| Preservative | 0.3% |
| Phase D | |
| Perfume oil | 0.2% |

Preparation of an Aqueous Shellac Solution/Dispersion

While stirring, the pH was adjusted to 3.3 with citric acid in 100 mL water. Then 1.3 g polyvinylpyrrolidone (PVPK30®) and 0.6 g guar hydroxypropyltrimonium chloride (CTFA name) (Jaguar C14S®) were added while stirring further. To the resulting solution was added 15 g purified, dewaxed, particulate shellac at approximately 13-15° C. and at less than 1000 rpm, and the solution was homogenized at 1450 rpm.

The shellac solution prepared in this way had a viscosity of 540 cP·s. Solutions and dispersions with higher viscosities and concentrations of shellac were prepared in the same way, varying the pH, the amount of shellac used and the amounts of the other components.

### EXAMPLE 3

Preparing a W/O Emulsion

A W/O emulsion was prepared from the following ingredients.

Phases A and B were prepared separately at 85° C. while stirring and combined at the same temperature and cooled to 40° C. while stirring. Then phase C was added to the total mixture while stirring.

| Phase A | |
|---|---|
| 1-Decane | 11.0% |
| Polyglycerol ester | 3.0% |
| Mineral oil | 5.0% |
| UV filter TiO₂/ZnO/benzophenone-3 | 3.5% |
| SiO₂ (monodisperse) (Merck) | 1.0% |
| Phase B | |
| Distilled water | q.s. ad 100 |
| Glycerol | 6.0% |
| Phase C | |
| Preservative | 0.2% |
| Perfume oil | 0.1% |

Phases A through C were prepared and mixed together in succession in this order at room temperature and homogenized.

| Phase A | |
|---|---|
| Distilled water | q.s. ad 100 |
| Crosspolymer | 1.0% |
| Glycerol | 2.0% |
| Phase B | |
| Triethanolamine | 1.0% |
| Phase C | |
| Perfume oil | 0.2% |
| Preservative, chamomile extract | 2.0% |
| Vitamin E | 0.5% |
| Vitamin C | 0.5% |
| Vitamin B₂ | 1.0% |

A shellac solution with a shellac content of 20%, prepared according to Example 2, was added as phase E in the amount of 15.0% to the mixture from Example 1 at 35° C. while stirring until the mixture was homogeneous.

### EXAMPLE 6

Sun Emulsion

A shellac solution with a shellac content of 10%, prepared according to Example 2, was added as phase D in the amount of 20.5% of the total composition to the mixture from Example 3 at 35° C. while stirring until the mixture was homogeneous.

### EXAMPLE 7

Skin Care Gel

A shellac solution or dispersion with a shellac content of 1%, prepared according to Example 2, was added as a component of phase C in the amount of 20.5% of the total composition to the mixture from Example 4 at ambient temperature while stirring until the mixture was homogeneous.

### EXAMPLE 8

| Lipstick | |
|---|---|
| Phase A | |
| Wax WM300/46 (CTFA name) | 70% |
| Castor oil | 12% |
| PPG 1-glyceryl oleostearate paraffin | 0.5% |
| Coloring agents | 4.5% |
| Phase B | |
| DMDM hydantoin | 0.6% |
| PPG 518MDI copolymer (CTFA name) | 1.0% |
| Water | q.s. ad 100 |
| Phase C | |
| Perfume oil | 0.5% |

Phase A was melted while stirring at 80° C. Phase A was added to phase B at 80° C. while stirring. Then the mixture was cooled to 50° C., and phase C was added at this temperature while stirring. Then a shellac dispersion with a shellac content of 50%, prepared according to Example 2, was added in the amount of 5% of the total composition while stirring until the mixture was homogeneous, and then the product was solidified.

### EXPERIMENTAL EXAMPLE 1

A product that was designated as “product A” was prepared according to Example 6. A similar product like that in Example 6 was prepared, but no shellac solution was added. This product was designated as “product B.”

For the purpose of testing while sunbathing, the two products were applied to the entire body of ten volunteers who were already pre-tanned.

Product A gave adequate UV protection on all volunteers. The product was applied only once each time over the period of the daily four-hour sunbath, and it had a sufficient water resistance with three interruptions by a 10-minute water bath each time.

Product B was applied at least twice by eight volunteers during the period of the daily four-hour sunbath to achieve the same UV protection. Its stability in water was not adequate.

1. A cosmetic product containing shellac, comprising a shellac content in the aqueous phase, where the pure shellac content is
in the range of 0.1 to 20 wt % in an O/W emulsion,
in the range of 0.1 to 15 wt % in a W/O emulsion,
in the range of 0.1 to 10 wt % in a hydrogel,
and the oily phase or the aqueous phase or both phases may contain additional active cosmetic ingredients or vehicles in the range of 1 to 20 wt %,
and all amounts are based on the weight of the total composition.

2. A cosmetic product containing shellac, wherein shellac is present in the aqueous phase, where the pure shellac content is
in the range of 0.1 to 20 wt % in an O/W emulsion,
in the range of 0.1 to 15 wt % in a W/O emulsion,
in the range of 0.1 to 10 wt % in a hydrogel,
and the oily phase or the aqueous phase or both phases contain additional cosmetic active ingredients or vehicles in the range of 1 to 20 wt %,
and all amounts are based on the weight of the total composition,
prepared by
a) preparing an emulsion or a hydrogel in the usual way by mixing a primary emulsion phase, containing the cosmetic active ingredients and additives provided therein, with a secondary emulsion phase, containing the cosmetic active ingredients and additives provided therein;
or mixing a gel-forming substance with water and adding the cosmetic active ingredients and additives provided therein; and
b) preparing a 5 wt % to 60 wt % aqueous shellac solution or dispersion by mixing pure shellac dry solids having a molecular weight of approximately 1000 with water in the presence of a water-soluble film-forming substance in the range of 0.1 to 3 wt % and a gel-forming substance that is stable in an acidic pH range in the range of 0.1 to 1 wt % plus an acid to adjust the pH to 2 to 4.2 while stirring at 150 to 1000 rpm for a period of 5 to 60 minutes at 20 to 45° C.; and
c) heating the emulsion or the gel from step a) to a temperature in the range of 35° C. to 45° C. while stirring; and
d) adding the shellac solution or dispersion from step b) while stirring at 150 to 1000 rpm for a period of 5 to 60 minutes at 35° C. to 45° C.; and then
e) cooling the resulting product to ambient temperature.

3. A product according to claim 2, wherein the concentration of the aqueous shellac solution or dispersion is 10 to 60 wt %.

4. A product according to claim 1 or 2, wherein the concentration of the aqueous shellac solution or dispersion is 15 to 60 wt %.

5. A product according to claim 4, wherein the concentration of the aqueous shellac solution or dispersion is 15 to 50 wt %.

6. A product according to claim 1, wherein it is a multiple emulsion of the W/O/W type or the O/W/O type.

7. A product according to claim 1 or 2, wherein the shellac content in the finished cosmetic product is in the range of 5 to 20 wt % in an O/W emulsion, in the range of 5 to 15 wt % in a W/O emulsion, and in the range of 3 to 10 wt % in a hydrogel.

8. A product according to claim 1 or 2, wherein the shellac content in the emulsion or the hydrogel is in the range of 0.5 to 8 wt %.

9. A product according to one of claims 1 through 8, wherein the viscosity of the shellac solution or dispersion is in the range of 500 to 100,000 cP·s.

10. A product according to claim 9, wherein the viscosity of the shellac solution or dispersion is in the range of 1000 to 100,000 cP·s.

11. A product according to claim 9, wherein the viscosity of the shellac solution or dispersion is in the range of 2500 to 100,000 cP·s.

12. A product according to one of claims 1 through 11, wherein it is in the form of a cream, lotion, sunscreen preparation, lipstick, make-up, mask, lip gloss, spray or gel.

13. A method of preparing a cosmetic product containing shellac, comprising the following steps:
a) preparing an emulsion or a hydrogel in the usual way by mixing a primary emulsion phase containing the active cosmetic ingredients and additives provided therein with a secondary emulsion phase containing the active cosmetic ingredients and additives provided therein, or mixing a gel-forming substance with water and adding the intended active cosmetic ingredients and additives; and
b) preparing a 5 to 60 wt % aqueous shellac solution or dispersion by mixing pure shellac dry solids having a molecular weight of approximately 1000 with water in the presence of a water-soluble film-forming substance in the range of 0.1 to 3 wt % and a gel-forming substance that is stable in the acid pH range in the range of 0.1 to 1 wt % and an acid for adjusting the pH to a level of 2 to 4.2 while stirring at 150 to 1000 rpm over a period of 5 to 60 minutes at 20 to 45° C.; and
c) heating the emulsion or the gel from step a) to a temperature in the range of 35 to 45° C. while stirring, and
d) adding the shellac solution or dispersion from step b) while stirring at 150 to 1000 rpm during a period of 5 to 60 minutes at 35 to 45° C.; and then
e) cooling the resulting product to ambient temperature.

14. A method according to claim 13, wherein the temperature is in the range of 5 to 15° C.

15. A method according to claim 13, wherein the pH is in the range of 2 to 3.5.
